(19) United States
(12) Patent Application Publication
(10) Pub. No.: US 2022/0032094 A1
(43) Pub. Date: Feb. 3, 2022

(54) Anti-Germ Breathing Device

(71) Applicant: Immanuel P. Maki, Boynton Beach, FL (US)

(72) Inventor: Immanuel P. Maki, Boynton Beach, FL (US)

(21) Appl. No.: 16/943,337

(22) Filed: Jul. 30, 2020

(51) Int. Cl. A62B 18/08; A62B 18/02; A62B 9/00; A62B 18/10; A62B 23/02; A62B 9/02

(52) U.S. Cl. CPC A62B 18/08; A62B 18/02; A62B 9/003; A62B 18/025; A62B 18/084; A62B 23/02; A62B 9/02; A62B 18/10

(57) ABSTRACT

An anti-germ breathing device is provided for a user, the device including a heating element housing an electric conduit; a one-way valve in communication with the heating element, the one-way valve having a first air filter and being structured and disposed for permitting entry of air from the atmosphere into the heating element when the user inhales through an inhale tube; a suction electric switch structured and disposed for connecting a battery with the electric conduit for heating the electric conduit when the user inhales; a cooling element housing a plurality of bulkheads; an air tube in fluid flow communication with each of the heating element and the cooling element; the plurality of bulkheads being sized and configured for evenly distributing air so that the air cools before it is inhaled by the user; and the inhale tube being in fluid flow communication with the cooling element such that the user inhales cooled

### FIELD OF THE INVENTION

This invention relates to a device for destroying the lipid bilayer of a coronavirus and, more particularly, a device for exposing heat to inhaled air in order to degrade the coronavirus.

### BACKGROUND

Scientists have described the structure of the coronavirus as follows. The nucleocapsid of coronavirus is formed from multiple copies of nucleocapsid protein which are bound to the positive sense single-stranded RNA genome in a continuous beads-on-string type confirmation. The lipid bilayer that envelopes the membrane proteins and the nucleocapsid, thus protecting the virus when outside. This lipid bilayer is a group of organic compounds consisting of fats and other substances of similar properties, not soluble in water, but in fat solvents and alcohol, 65%.

The device disclosed herein aims to destroy the coronavirus with heat. At a given temperature the lipid bilayer can exists liquid or solid state. When the temperature rises, all lipids are destroyed as when diluted with a solvent or alcohol. When the protective layer, the lipid bilayer, is differentiated from the nucleocapsid it loses its protective layer and disperses and breaks down on its own. The same process also occurs when washing hands with soap and warm water that CDC has recommended.

### SUMMARY OF THE INVENTION

An anti-germ breathing device for a user, the device including a heating element housing an electric conduit; a one-way valve in communication with the heating element, the one-way valve having a first air filter and being structured and disposed for permitting entry of air from the atmosphere into the heating element when the user inhales through an inhale tube; a suction electric switch structured and disposed for connecting a battery with the electric conduit for heating the electric conduit when the user inhales; a cooling element housing a plurality of bulkheads; an air tube in fluid flow communication with each of the heating element and the cooling element; the plurality of bulkheads being sized and configured for evenly distributing air so that the air cools before it is inhaled by the user; and the inhale tube being in fluid flow communication with the cooling element such that the user inhales cooled air.

An anti-germ breathing device for a user, the device including a heating element housing an electric conduit; a one-way valve in communication with the heating element, the one-way valve having a first air filter and being structured and disposed for permitting entry of air from the atmosphere into the heating element when the user inhales through an inhale tube; a suction electric switch structured and disposed for connecting a battery with the electric conduit for heating the electric conduit when the user inhales; a plurality of straps for securing the heating element and cooling element to the user; a cooling element housing a plurality of bulkheads; an air tube in fluid flow communication with each of the heating element and the cooling element; the plurality of bulkheads being sized and configured for evenly distributing air so that the air cools before it is inhaled by the user; and the inhale tube being in fluid flow communication with the cooling element such that the user inhales cooled air.

### BRIEF DESCRIPTION OF THE DRAWINGS

For a fuller understanding of the nature of the present invention, reference should be made to the following detailed description, taken in conjunction with the accompanying drawings in which:

FIG. 1 illustrates a rear perspective view of the anti-germ breathing device;

FIG. 2 illustrates a front perspective view thereof with an air filter;

FIG. 3 illustrates a front perspective view thereof with a supporting screen when the air filter and the supporting fixture covering the anti-germ breathing device is removed;

FIG. 4 illustrates a front perspective view of the anti-germ breathing device with the supporting screen, the air filter and the supporting fixture covering the anti-germ breathing device removed therefrom;

FIG. 5 illustrates a front elevational view of the inhale component attached to a heating element;

FIG. 6A illustrates a side elevational view of the suction electric switch inside the oval of the oval-shaped heating element;

FIG. 6B illustrates a cross-sectional view taken along line A-A of FIG. 6A and showing the mounting of the diaphragm;

FIG. 7A illustrates a side elevational view of the inside of the heating element;

FIG. 7B illustrates the vertical elevational view of the anti-germ breathing device;

FIG. 8 illustrates the supporting fixture;

FIG. 9 illustrates the front perspective view of the anti-germ breathing device attached to a face mask;

FIG. 10 illustrates a side elevational view of the extension inhale tube attached to another face mask; and

FIG. 11 illustrates a side elevational view of the anti-germ breathing device with the extension inhale tube attached and carried as a backpack.

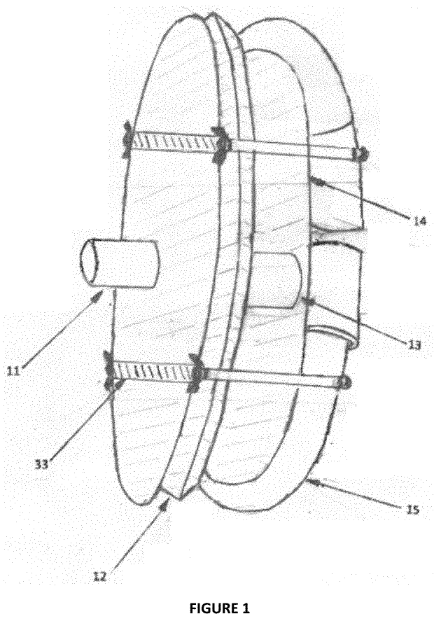

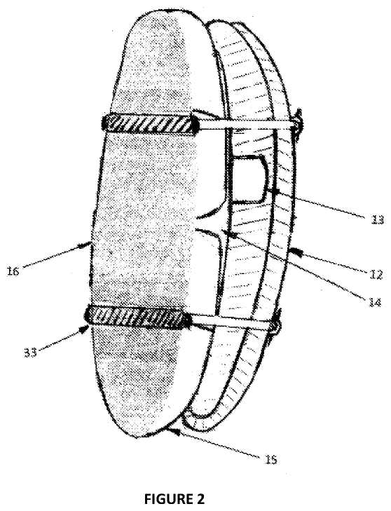

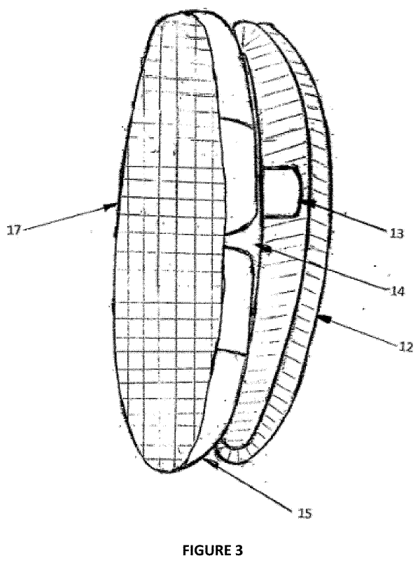

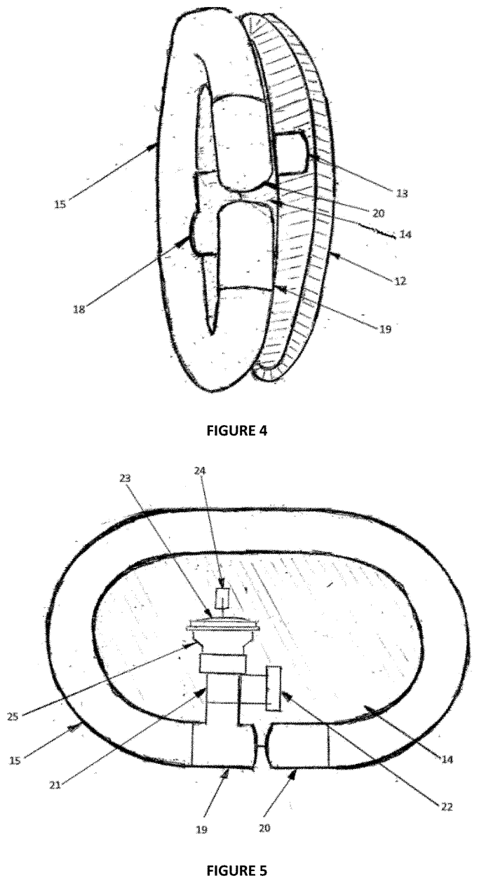

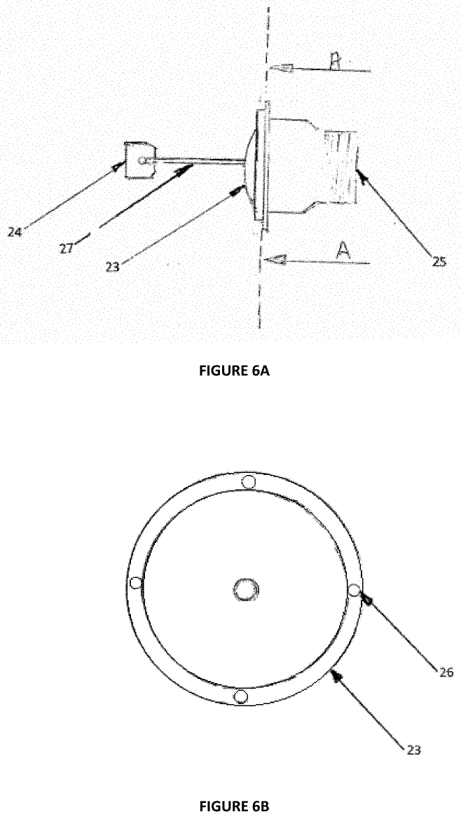

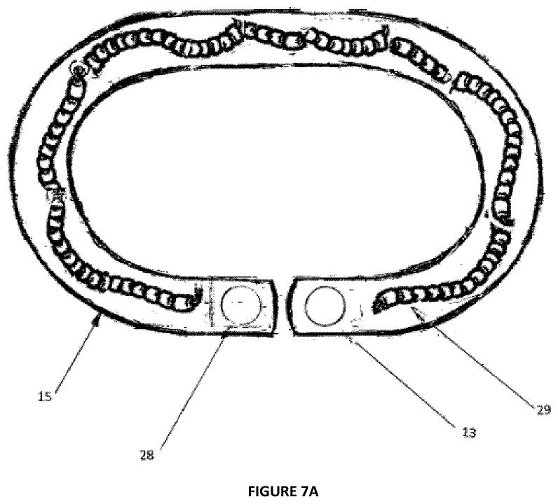

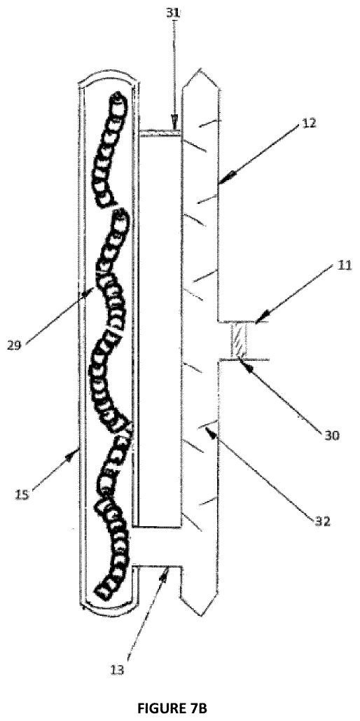

Like reference numerals refer to like parts throughout the several views of the drawings.

### DETAILED DESCRIPTION

An anti-germ breathing device is shown and described herein and is generally indicated as 10. Below is a parts listing relating to the device 10:
11—inhale tube
12—cooling element
13—air tube between the cooling element and the heating element
14—air and heat insulating plate
15—heating element
16—first air filter
17—supporting screen
18—inhale component
19—air L-connector, lets the air flow from outside thru the filter into the heating element
20—hot air L-connector into the cooling element
21—Y-connector with a one-way valve and suction electric switch
22—inhale one-way valve
23—suction switch diaphragm
24—suction electric switch
25—suction switch diaphragm mounting
26—fastener of the suction switch diaphragm
27—draw connector
28—air canal into the heating element
29—electric conduit inside of a string of fish spine beads
30—second air filter
31—supporting bar
32—air flow directing bulkheads
33—supporting fixture of the anti-germ breathing device
34—connecting metal bar
35—screw hole
36—wing nut
37—nut
38—connecting screw
40—face mask
41—exhale one-way valve
42—inhale tube extension
43—carrying straps
44—battery

The anti-germ breathing device 10 utilizes electricity to heat the inhaled air to 100° F. (40° C.) to 200° F. (90° C.) or even more. The temperature is adjusted with a resistor that is located on top of the lithium battery 44. The battery 44 is carried by the user either at the user's waist or over the shoulder bag.

The user puts on a mask 40 over his or her face. The breathing device 10 is fitted in front of the mask 40. When the user starts to inhale, a one-way valve 22 connected to the conduit opens. This inhale one-way valve 22 is connected to a suction electric switch 24 that turns on the electricity to an electric conduit 29. This electric conduit 29 is situated in the inhale tube 11 and it heats the conduit 29 to effectively burn viruses and bacteria from the inhaled air.

A human's inhale breath generally takes 3 to 4 seconds and when the user starts to exhale the one-way inhale valve 22 closes and disconnects the electricity flow to the conduit 29 which starts to cool down. The air in the heating element 15 flows to the cooling element 12 during the next inhaling and through the filter to the lungs of the inhaler.

The user's inhalation is the power that starts the operation of the device 10. The power source of the electrical system is a battery, such as a 24V lithium battery, with two poles. An electric wire from the battery goes into the inhale tube 11 and is connected to the electric conduit. When the inhale one-way valve opens, the electric is connected to the electric conduit by the switch. The battery functions only at a time that the inhale lasts 3-4 seconds at a time. An adjusting resistor controls the temperature of the electric conduit in the inhale tube 11. By turning the resistor, the temperature of the electric conduit 29 in the inhale tube 11 can be regulated. In one embodiment, the resistor is on the battery.

In operation, the user puts a mask on and starts to inhale air through the inhale tube 11 and it opens the inhale one-way air valve 22. A low vacuum forms which forces the suction switch diaphragm 23 to move inwards to the suction switch diaphragm mounting 25. The draw connector 27 is connected to the suction switch diaphragm 23 at one end and to the suction electric switch 24 at the opposite end. Simultaneously, the suction electric switch 24 turns on the electricity to the electric conduit 29 located inside the heating element 15. In one embodiment, the electric conduit 29 is formed in a string of fish spine beads. It heats the air inside the heating element 15 and destroys the coronaviruses and bacteria. The required temperature is reached by using the thermal-electric resistor located on top of the battery 44.

The inhaled air then flows into the cooling element 12 which has several bulkheads 32 inside where the inhale air flows. The bulkheads 32 guide the inhaled air to be distributed evenly so that the inhaled air cools before it is inhaled into the lungs.

The inhale tube 11 includes an air filter 30 for collecting any possible residual particles from the heating element 15 due to the high temperatures. The exhaled air is directed by a one-way exhale valve 41 in the face mask 40 into the air outside on top of the cooling element 12 of the anti-germ breathing device 10 for additional cooling.

The device 10 has two main parts, the heating element 15 and the cooling element 12, equal in size, external width and height. The inhaled air first enters into the heating element 15 and next into the cooling element 12 with bulkheads 32.

The inhaled air first flows through the air filter 16 and through the oval tube. The inhaled air then flows into the plated cooling element 12 and through another filter 30 into the lungs. The air flow is controlled by two one-way valves. The first one-way-valve is located in the inhale fitting of the oval heating tube underneath the first air filter 16. The second air filter 30 is in the fitting of the inhale tube 11 connected to the protective mask. When the user inhales air the inhale one-way valve inside the oval of the heating element 15 opens. The exhale one-way valve 41 in the face mask stays closed and the inhaled air stays in a closed space about 3 to 5 seconds. When the exhale begins the exhale one-way valve 41 opens in the face mask and the inhale one-way valve stays closed.

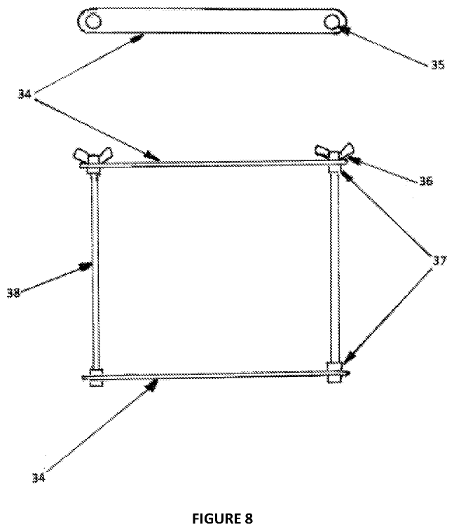

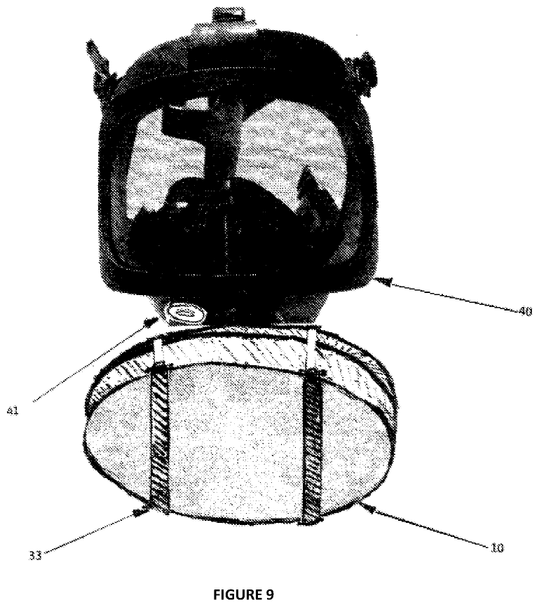

Referring specifically to FIG. 8, two identical supporting fixtures, 33, secure the anti-germ breathing device. The supporting fixture has two light metal bars, 34, like aluminum, with two screw holes, 35, one at each end. The connecting screw, 38, goes through one hole, 35, of the metal bar, 34, and fastened by a nut, 37, and through another hole, 35, in another metal bar, 34, secured with a nut, 37, and the wing nut, 36, at the top. The other ends of the metal bars, 34, are assembled like the first forming a rectangle. When the air filter needs to be cleaned or replaced the four wing nuts can be unscrewed. Then the metal bars come loose and are easily removed as the whole supporting fixtures. The assembly is to screw the four wing nuts back on when the air filter is cleaned or replaced. In one embodiment, a supporting strap for the anti-germ breathing device can be easily attached with hooks to the outer screws of the supporting fixture on the both sides of the anti-germ device. Then the strap is secured at the back of the user's head.

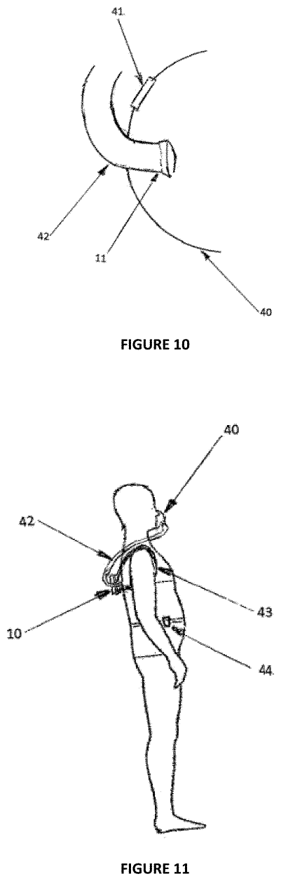

Referring now to FIGS. 10 and 11, in accordance with one embodiment, the anti-germ breathing device 10 may be formed with straps 43, such as straps 43 forming part of a backpack, to be worn by a user. The device 10 is attached with four light straps 43 with hook and loop fasteners. The inhale extension tube 42 is attached between the cooling element 12 and the face mask. FIG. 11 further illustrates the battery 44 carried on a waist belt.

Unless otherwise defined, all terms (including technical and scientific terms) used herein have the same meaning as commonly understood by one of ordinary skill in the art to which this subject matter belongs. It will be further understood that terms, such as those defined in commonly used dictionaries, should be interpreted as having a meaning that is consistent with their meaning in the context of the specification and relevant art and should not be interpreted in an idealized or overly formal sense unless expressly so defined herein. For brevity and/or clarity, well-known functions or constructions may not be described in detail herein.

The term “exemplary” is used herein to mean serving as an example, instance, or illustration. Any aspect or design described herein as “exemplary” is not necessarily to be construed as preferred or advantageous over other aspects or designs. Similarly, examples are provided herein solely for purposes of clarity and understanding and are not meant to limit the subject innovation or portion thereof in any manner.

The terms “for example” and “such as” mean “by way of example and not of limitation.” The subject matter described herein is provided by way of illustration for the purposes of teaching, suggesting, and describing, and not limiting or restricting. Combinations and alternatives to the illustrated embodiments are contemplated, described herein, and set forth in the claims.

For convenience of discussion herein, when there is more than one of a component, that component may be referred to herein either collectively or singularly by the singular reference numeral unless expressly stated otherwise or the context clearly indicates otherwise. For example, components 38 (plural) or component 38 (singular) may be used unless a specific component is intended. Also, the singular forms “a,” “an,” and “the” are intended to include the plural forms as well, unless expressly stated otherwise or the context indicates otherwise.

It will be further understood that the terms “includes,” “comprises,” “including,” and/or “comprising” specify the presence of stated features, steps, operations, elements, and/or components, but do not preclude the presence or addition of one or more other features, steps, operations, elements, components, and/or groups thereof unless explicitly stated otherwise or the context clearly requires otherwise. The terms “includes,” “has” or “having” or variations in form thereof are intended to be inclusive in a manner similar to the term “comprises” as that term is interpreted when employed as a transitional word in a claim.

It will be understood that when a component is referred to as being “connected” or “coupled” to another component, it can be directly connected or coupled or coupled by one or more intervening components unless expressly stated otherwise or the context clearly indicates otherwise.

The term “and/or” includes any and all combinations of one or more of the associated listed items. As used herein, phrases such as “between X and Y” and “between about X and Y” should be interpreted to include X and Y unless expressly stated otherwise or the context clearly indicates otherwise.

Terms such as “about”, “approximately”, and “substantially” are relative terms and indicate that, although two values may not be identical, their difference is such that the apparatus or method still provides the indicated or desired result, or that the operation of a device or method is not adversely affected to the point where it cannot perform its intended purpose. As an example, and not as a limitation, if a height of “approximately X inches” is recited, a lower or higher height is still “approximately X inches” if the desired function can still be performed or the desired result can still be achieved.

While the terms vertical, horizontal, upper, lower, bottom, top and the like may be used herein, it is to be understood that these terms are used for ease in referencing the drawing and, unless otherwise indicated or required by context, does not denote a required orientation.

The different advantages and benefits disclosed and/or provided by the implementation(s) disclosed herein may be used individually or in combination with one, some or possibly even all of the other benefits. Furthermore, not every implementation, nor every component of an implementation, is necessarily required to obtain, or necessarily required to provide, one or more of the advantages and benefits of the implementation.

Conditional language, such as, among others, “can”, “could”, “might”, or “may”, unless specifically stated otherwise, or otherwise understood within the context as used, is generally intended to convey that certain embodiments preferably or optionally include certain features, elements and/or steps, while some other embodiments optionally do not include those certain features, elements and/or steps. Thus, such conditional language indicates, in general, that those features, elements and/or step may not be required for every implementation or embodiment.

Those skilled in the art will recognize many modifications may be made to the implementation(s) disclosed herein without departing from the scope or spirit of the claimed subject matter. The subject matter described above is provided by way of illustration only and should not be construed as limiting. Furthermore, the claimed subject matter is not limited to implementations that solve any or all disadvantages noted in any part of this disclosure. Various modifications and changes may be made to the subject matter described herein without following the exemplary embodiments and applications illustrated and described, and without departing from the spirit and scope of the following claims.

What has been described above includes examples of aspects of the claimed subject matter. It is, of course, not possible to describe every conceivable combination of components or methodologies for purposes of describing the claimed subject matter, but one of ordinary skill in the art may recognize that many further combinations and permutations of the disclosed subject matter are possible. Accordingly, the disclosed subject matter is intended to embrace all such alterations, modifications and variations that fall within the spirit and scope of the appended claims.

Although the subject matter presented herein has been described in language specific to components used therein, it is to be understood that the appended claims are not necessarily limited to the specific component or characteristic thereof described herein. Rather, the specific components and characteristics thereof are disclosed as example forms of implementing the claims.

What is claimed is:

1. An anti-germ breathing device for a user, the device comprising:
a heating element housing an electric conduit;
a one-way valve in communication with the heating element, the one-way valve having a first air filter and being structured and disposed for permitting entry of air from the atmosphere into the heating element when the user inhales through an inhale tube;
a suction electric switch structured and disposed for connecting a battery with the electric conduit for heating the electric conduit when the user inhales;
a cooling element housing a plurality of bulkheads;
an air tube in fluid flow communication with each of the heating element and the cooling element;
the plurality of bulkheads being sized and configured for evenly distributing air so that the air cools before it is inhaled by the user; and
the inhale tube being in fluid flow communication with the cooling element such that the user inhales cooled air.

2. The anti-germ device as recited in claim 1 further comprising a one-way exhale valve.

3. The anti-germ device as recited in claim 2 further comprising a mask.

4. The anti-germ device as recited in claim 1 further comprising a second air filter in the inhale tube.

5. The anti-germ device as recited in claim 3 further comprising an inhale extension tube attached between the cooling element and the face mask.

6. The anti-germ device as recited in claim 1 further comprising a suction switch diaphragm that is structured and disposed to move inwards when the user inhales for selectively operating the suction electric switch.

7. An anti-germ breathing device for a user, the device comprising:
a heating element housing an electric conduit;
a one-way valve in communication with the heating element, the one-way valve having a first air filter and being structured and disposed for permitting entry of air from the atmosphere into the heating element when the user inhales through an inhale tube;
a suction electric switch structured and disposed for connecting a battery with the electric conduit for heating the electric conduit when the user inhales;
a plurality of straps for securing the heating element and cooling element to the user;
a cooling element housing a plurality of bulkheads;
an air tube in fluid flow communication with each of the heating element and the cooling element;
the plurality of bulkheads being sized and configured for evenly distributing air so that the air cools before it is inhaled by the user; and
the inhale tube being in fluid flow communication with the cooling element such that the user inhales cooled air.

8. The anti-germ device as recited in claim 7 further comprising a one-way exhale valve.

9. The anti-germ device as recited in claim 8 further comprising a mask.

10. The anti-germ device as recited in claim 7 further comprising a second air filter in the inhale tube.

11. The anti-germ device as recited in claim 9 further comprising an inhale extension tube attached between the cooling element and the face mask.

12. The anti-germ device as recited in claim 7 further comprising a suction switch diaphragm that is structured and disposed to move inwards when the user inhales for selectively operating the suction electric switch.
